(19) United States
(12) Patent Application Publication
(10) Pub. No.: US 2009/0099474 A1
(43) Pub. Date: Apr. 16, 2009

(54) System and method for combined bioelectric sensing and biosensory feedback based adaptive therapy for medical disorders

(76) Inventors: Jaime A. Pineda, San Diego, CA (US); Erik Viirre, San Diego, CA (US)

Correspondence Address: LIU & LIU, 444 S. FLOWER STREET SUITE 1750, LOS ANGELES, CA, 90071, US

(21) Appl. No.: 12/286,895

(22) Filed: Oct. 1, 2008

Related U.S. Application Data
(63) Continuation-in-part of application No. 12/215,385, filed on Jun. 25, 2008.
(60) Provisional application No. 60/997,236, filed on Oct. 1, 2007.
(60) Provisional application No. 61/001,209, filed on Oct. 30, 2007.

(51) Int. Cl. A61B 5/04

(52) U.S. Cl. 600/545

(57) ABSTRACT

A system and method for adaptive therapeutic intervention to effect real-time changes in the behavioral profile of an individual to facilitate the effectiveness of combined bioelectric sensing and biosensory based therapy for specific disorders. The system and method induces a temporary physiological state-of-mind (e.g., increased relaxation) to effect persistent changes to the cognitive-emotive profile of the individual (e.g., reduce internal tinnitus). The sense- and mental state-awareness responses, integrated into a two-way (i.e., bi-directional) feedback system using a dynamic interface with intelligently controlled thresholds. The invention takes into account details of multi-variate and nonlinear dynamics and database templates to more accurately compute the user's “state-of-mind.” It then utilizes this “state-of-mind” to drive therapeutic and non-therapeutic stimulus intervention. By way of a “combinatorial stimulation sequence” approach that uses customized sounds, the present invention creates a fine-tuned and well-controlled process. The significance of this interactivity is a prolonged change in the individual's cognitive-emotive profile.

This application claims the priority of Provisional Patent Application No. 60/997,236 which was filed Oct. 1, 2007 and Provisional Patent Application No. 61/001,209, which was filed Oct. 30, 2007; this application is a continuation-in-part application of U.S. patent application Ser. No. 12/215,385, which was filed Jun. 25, 2008. These earlier applications and all patent documents and other publications disclosed herein below are fully incorporated by reference, as if fully set forth herein.

### BACKGROUND OF THE INVENTION

1. Field of the Invention

The present invention generally relates to medical apparatus, in particular systems and methods for combined bioelectric sensing (e.g., neurological feedback) and biosensory feedback based therapy, and more particularly application of such system and method for adaptive therapy of disorders, such as central auditory processing disorders.

2. Description of Related Art

Central auditory processing disorders (CAPDs) occasioned by organic or traumatic events can cause functional disruption to cognitive-emotive circuits in areas of the brain that are then reflected in difficulties understanding conversations in noisy environments; problems following complex directions; difficulty learning new vocabulary words or foreign languages; difficulty localizing sounds; auditory discrimination; temporal aspects of audition including: temporal resolution; temporal masking, temporal integration and temporal ordering; auditory performance with competing acoustic signals; and auditory performance with degraded signals. CAPDs are also characterized by a loss of control, initiation, cessation, control of action, cognitive estimation, cognitive flexibility, deficits in the response to novelty, goal-directed behaviors and the ability to sequence. Many of these deficits are reflected in the symptoms of specific malfunctions such as chronic fatigue, pain, tinnitus, epilepsy, depression, sleep disorders, and addiction, among others. Recent scientific evidence has shown that it is possible to temporarily activate or deactivate specific cognitive-emotive brain circuits via Transcranial Magnetic Stimulation (TMS) in order to ameliorate, prevent, and possibly cure such brain malfunctions, and hence improve the overall health of the individual. However, TMS is currently a rather invasive procedure with little applicability to a general population that can include children. Instead, a sound based approach having similar outcomes would provide a better vector for providing therapy for CAPDs.

A customized sound therapy (CST) approach has been developed by the inventors to address the problem of tinnitus. That methodology can be used to address a variety of similar disorders involving dysregulation of auditory processing in the brain. Tinnitus is a debilitating condition defined as the sensation of “ringing in the ears” in the absence of external stimuli. The American Tinnitus Association reports that approximately 36 million Americans have some form of tinnitus, with over 12 million Americans suffering from tinnitus so severe that quality of life is seriously compromised. The United States Veterans Administration alone spends over $110 million annually on tinnitus related disability benefits for former U.S. Armed Forces personnel. Over one-third of Americans over the age of 65 are affected by tinnitus, and thus, it is also the tenth most common presenting complaint among the elderly in primary care. Given the aging demographics of the U.S., the prevalence of this condition is only expected to rise in coming years.

Despite the societal impact of tinnitus, there is currently no effective treatment available in the marketplace, and a definitive cure for tinnitus continues to represent a major unmet medical need. Various drug therapies, including antidepressants and sedative agents, only indirectly manage secondary effects of the disease. Auditory masking devices are also available that temporarily decrease the perception of tinnitus by presenting an artificial neutral “white noise,” but these instruments only transiently control symptoms and do not address the underlying pathology. Tinnitus retraining therapy with concomitant cognitive psychotherapy is also a popular treatment modality popularized in recent years, but has not demonstrated reproducible efficacy, requires an intensive treatment course of two years for completion, and is costly—on the order of $3,000 USD or more.

It is known in the literature that complex interactions exist between stress and the exacerbation of tinnitus. Neurofeedback training allows a person to reduce his or her physiological and psychological reactivity to stress and is thus considered a viable tinnitus treatment option. Neurofeedback devices measure changes in the EEG and display these effects for the patient to see. The patient then attempts to exert volitional control over these measured parameters. Neurofeedback by itself has been shown to be effective. The use of brain signals for monitoring, actuation and controlling states of mind related to stress and anxiety can provide a complementary therapeutic role when implemented simultaneously in a real-time environment with customized sound therapy. These types of brain signal/resource interactions are representative of an intelligent control system, which increases the degrees of freedom of action that a user has over his condition.

However, the use of an intelligent control system to control stress in real time, which is combined with a sound-based tinnitus treatment, is currently unavailable. Though there have been suggestions on the use of bioelectric signals to control content and levels of performance in other environments such as video games, such technologies exhibit lack of degrees of freedom in controllability. An example is that in the prior art rapid bi-directional control is non-existent. It also does not take full advantage of direct brain control, which is a completely new means of controlling a brain signal. Furthermore, while existing prior art uses spontaneous bioelectric signals, such as heart rate (HR), electrooculogram (EOG), galvanic skin response (GSR), and the electroencephalogram (EEG), it focuses on a single outcome function, which relates the signal to a single outcome event. The inference of such an outcome from a single signal fails to take into account the dynamics of the different brain and peripheral signals, singularly, or in combination. Although the use of any of these signals can effect, as a substitute for, the physiological function of human interaction with the environment, one's control over the resources of the environment is essentially devoid of the dynamics of the state of the mind of the user. Such state of the mind dynamics is considered to be reflected in the non-linear combination of different brain and peripheral signals. Bioelectric signals provide a window into the complex dynamics of brain activity related to sensation, motor, and cognitive behavior. The use of brain signals to assess sensorimotor (“sense awareness”) and psychological (“mental state awareness”) dimensions provides the front end to such an adaptive system. Furthermore, it is possible to similarly assess the bodily environment (e.g., HR, GSR, and EOG) in which a behavior occurs (“context awareness”). Hence, the multi-dimensional, non-linear combination of sense, mental state-, and context-awareness information provides a more realistic bioelectric snapshot of an individual's “state-of-mind.” Finally, current art does not typically utilize wireless information transmission, nor bioelectric signals in an active, controlling way, and does not extract event-related signals from the ongoing, spontaneous EEG.

Accordingly, it would be desirable to have a system that incorporates non-linear dynamics as part of an intelligent controller to enable dynamic mapping between bioelectric signals and outcome events. Furthermore, it would be desirable for this system to recognize the functional significance of the various relevant components of the signals measured in the form of bioelectric patterns. It would also be desirable for it to be wireless, volitional, and to use the full complement of information present in the bioelectric input. The present invention, a combined stress and anxiety-reducing neurofeedback process coupled with customized sound therapy addresses these fundamental attributes, adding improved efficiency and efficacy to the treatment of tinnitus and other CAPDs.

Therapeutic and even non-therapeutic treatments that dynamically and in real time adapt to a person's psychological stress responses to the intervention provide a level of sophistication and care that is more sensitive and effective than traditional methods of treatment. It is known that brain bioelectric signals provide a window into the complex dynamics of brain activity related to sensation, motor, and cognitive-emotive behavior. Because of recent improvements in biological sensor technology, signal processing methodology, pattern recognition techniques, and high-speed computational algorithms, the development and use of techniques to characterize a temporary, physiological “state-of-mind” in real time have improved considerably. Similarly, the use of neurofeedback techniques to induce a temporary state-of-mind and to effect changes in an individual's stress response and cognitive-emotive profile, such as in treating tinnitus, has been well documented.

U.S. Pat. No. 7,081,085 to Viirre et al., entitled “EEG Feedback Controlled Sound Therapy for Tinnitus” teaches a method for treating tinnitus by habituation through use of neurological feedback, comprising the steps of connecting a subject through a set of attached headphones to an electronic sound, and generating an EEC signature of the subject's brain activity in response to the presented sound, sound using the customized sound to stimulate the auditory system while the brain activity is recorded, wherein the computer continuously monitors for the feedback signatures and drives the sound stimuli appropriately.

However, current sound-based therapies for CAPDs do not work by controlling levels of external therapy, lack rapid bi-directional control, and are relatively insensitive to the user's stress levels and cognitive-emotive profile. While they primarily deal with reducing or eliminating symptoms, they are not focused on improving individual wellness. Additionally, existing technologies are psychologically demanding and require long periods of time to effect the desired therapeutic changes.

What is needed is a means and a method to induce a temporary state-of-mind using low-level, sound-based therapy and stress reduction to effect persistent changes in the auditory profile of individuals suffering with CAPDs. Such a system should rapidly recognize the functional significance of the mental and brain function. Moreover, such a system could be used, though not limited, to enhance cognition, enhance wellness, improve quality of medical care, reduce the time to therapeutic effectiveness, and diminish the intervention time necessary to ameliorate specific disorders such as chronic fatigue, pain, tinnitus, depression, sleep disorders, addiction, anxiety, post traumatic stress syndrome, obsessive compulsive disorder, eating disorders, motor skills disorders, communication disorders, attention deficit hyperactivity disorder, autism, dissociative disorders, and impulse control disorders.

### SUMMARY OF THE INVENTION

The present invention relates to a system and method for adaptive therapeutic intervention to effect real-time changes in the behavioral profile of an individual to facilitate the effectiveness of combined bioelectric sensing and biosensory based therapy for specific disorders. The present invention comprises a system and method for inducing a temporary physiological state-of-mind (e.g., increased relaxation) to effect persistent changes to the cognitive-emotive profile of the individual (e.g., reduce internal tinnitus). Capable of rapidly recognizing the functional significance of the mental and brain function, the invention represents a unique approach to neurofeedback and “mental-state” therapy. The invention makes possible sensitive management of types and levels of therapeutic and non-therapeutic interventions. The sense- and mental state-awareness responses, integrated into a two-way (i.e., bi-directional) feedback system using a dynamic interface with intelligently controlled thresholds. The invention takes into account details of multi-variate and nonlinear dynamics and database templates to more accurately compute the user's “state-of-mind.” It then utilizes this “state-of-mind” to drive therapeutic and non-therapeutic stimulus intervention. By way of a “combinatorial stimulation sequence” approach that uses customized sounds, the present invention creates a fine-tuned and well-controlled process. The significance of this interactivity is a prolonged change in the individual's cognitive-emotive profile.

In one aspect, the present invention relates to a system and method for adaptive therapeutic intervention to effect real-time relaxation changes in the behavioral profile of an individual to facilitate the effectiveness of customized sound therapy for specific central auditory processing disorders. In particular, this aspect of the invention relates to a system and method that involve the use of neurofeedback techniques using bioelectric fields and characterization of bioelectric activity for producing real-time, adaptive changes in the behavioral profiles of individuals while undergoing customized sound therapy for a variety of disorders. It combines recently developed advances in neurophysiological techniques into a system suitable for real time adaptive therapy that will enhance wellness, speed up and improve the quality of care, and minimize intervention for specific disorders such as chronic fatigue, pain, tinnitus, depression, sleep disorders, addiction, anxiety, post traumatic stress syndrome, obsessive compulsive disorder, eating disorders, motor skills disorders, communication disorders, attention deficit hyperactivity disorder, autism, dissociative disorders, and impulse control disorders.

In one embodiment, the present invention integrates a combinatorial recording approach with a combinatorial sound-based stimulation approach, which enables real time, adaptive changes. It comprises a portable headset that includes a number of EEG recording sensors (e.g., dry, non-contact electrodes suitable for use without affecting skin condition) and a set of software tools that allow for real-time, bi-directional feedback of EEG signals. The headset device captures bioelectric signals. Through neurofeedback of the stress response combined with customized sound therapy specific brain and mental states can be induced in which individuals experience behavioral changes, such as improvement in their tinnitus experience. Real time assessment of bioelectric indices (both peripheral and central) is used to compute a multi-dimensional “state-of-mind” of the individual that reflects the assessment of current sensorimotor (“sense awareness”) and psychological (“mental state awareness”) states and their boundary conditions. This can then be used to provide direct feedback to the user or to adjust the duration, timing, and pulsatile nature of the customized sound therapy.

The inventive system is real-time and adaptive to the changing state of the individual. Hence, a course of treatment could involve an individual learning through a process of instrumental conditioning how to reduce levels of anxiety and stress, adjust the necessary level of customized sound therapy stimulation and gradually decrease or modify such stimulation over time. The outcome would be a change in the individual's cognitive-emotive profile for tinnitus. The present invention can be used alone or in combination with other interventions to produce these desired changes.

In one embodiment, a personal sound player (PSP) is used in the present invention to provide an intelligent control interface to monitor, record, and transform bio-signals, as well as interact with built-in software implemented processes for customized sound therapy. The interface comprises means for acquiring the bioelectric signals of a user, which are converted into a digital stream, processed and combined to define a cognitive-emotive profile as well as a “state of mind” of the user. This cognitive-emotive profile is used to drive the relaxation neurofeedback and interact with the customized sound therapy. Incorporating microprocessor-based software and database capabilities, the interface dynamically maps the cognitive-emotive profile onto multiple functions, which are adaptable for actuating microprocessor commands. In conjunction with other standard input devices such as mouse, keyboard, or joystick, the intelligent control interface of the present invention provides a user with control over the sound experience that may be creating problems.

In addition to providing such control the interface is adaptable to wirelessly map bioelectric signals into microprocessor commands in real time. It further enables closed- and open-loop feedback without the need for the constant monitoring of the input. The invention incorporates adaptive pattern recognition functions (e.g., by way of a software implemented module) that allow for automated learning, whereby the system learns to recognize a user's specific cognitive-emotive profile, as it changes over time. It handles spontaneous EEG rhythms, particularly those measured over the sensorimotor cortex, time-locked responses to external events, or event-related potentials (ERPs), steady state visual evoked responses in a rapid, bi-directional way, as well as autonomic measures of bodily states (e.g., HR, GSR, and EOG). The device enables the user to learn to control the magnitude and effect of bioelectric signals and to do so within a short period of time. By controlling these signals, for example, in producing or blocking them, the user can dynamically alter levels of anxiety and stress that make customized sound therapy more effective.

In one embodiment, the PSP device is structured and configured to provide a mapping function, which is comprised of inputs from various types of bioelectric signals that are recorded. A combinatorial software implemented function captures the interdependencies among the signals, which are then associated with the particular microprocessor commands. The extracted signal obtained from the user can be analyzed and used for control in a variety of ways. For example, the different dimensions of the signal can be decomposed, analyzed, and configured with respect to normative patterns in a database. Further, individual differences in baseline levels and in the degree of control that users learn to exert over their own signals can be analyzed and resolved by the learning module (e.g., algorithms). The result is a highly adaptive capability of the present system that can be refined over time.

Thus, the PSP device of the present system represents a unique, novel, more natural, intuitive, and hands-free means of controlling stress and applying sound-based therapy to tinnitus and other similar disorders of the central auditory system. Taking into account the non-linear details of neurodynamics and reflecting the user's mood and state of mind, the PSP guides a shapeable experience and defines how brain resources are best utilized.

### BRIEF DESCRIPTION OF THE DRAWINGS

For a fuller understanding of the scope and nature of the invention, as well as the preferred mode of use, reference should be made to the following detailed description read in conjunction with the accompanying drawings. In the following drawings, like reference numerals designate like or similar parts throughout the drawings.

FIG. 1 is a schematic diagram illustrating the NeuroTherapeutic Wellness System in accordance with one embodiment of the present invention.

FIG. 2 is a flow diagram of the recording of brain signals in accordance with one embodiment of the present invention.

FIG. 3 is a schematic depicting the decomposition and analysis of brain signals in accordance with one embodiment of the present invention.

FIG. 4 is a flow diagram of the learning and pattern recognition analysis of brain signals in accordance with one embodiment of the present invention.

FIG. 5 is a flow diagram of the computer interface and closed-loop feedback analysis of brain signals in accordance with one embodiment of the present invention.

FIG. 6 is a schematic depiction of a neural network in accordance with one embodiment of the present invention.

FIG. 7 is a schematic depiction of a computational algorithm implementation of the process for manipulating input signals in accordance with one embodiment of the present invention.

FIG. 8 is a schematic diagram of a controlling system for driving neural activity changes, in accordance with one embodiment of the present invention.

FIG. 9 is a perspective view of an audio device that can be implemented with the inventive system in accordance with one embodiment of the present invention.

### DETAILED DESCRIPTION OF EMBODIMENTS OF THE INVENTION

The present description is of the best presently contemplated mode of carrying out the invention. This description is made for the purpose of illustrating the general principles of the invention and should not be taken in a limiting sense. The scope of the invention is best determined by reference to the appended claims.

The detailed descriptions of the system and process of the present invention are presented in terms of schematics, functional components, methods or processes, symbolic or schematic representations of operations, functionalities and features of the invention. These descriptions and representations are the means used by those skilled in the art to most effectively convey the substance of their work to others skilled in the art. The invention may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network or other data transmission medium. In a distributed computing environment, program modules and other data may be located in both local and remote computer storage media including memory storage devices. A software implemented method or process is here, and generally, conceived to be a self-consistent sequence of steps leading to a desired result. These steps require physical manipulations of physical quantities. Often, but not necessarily, these quantities take the form of electrical or magnetic signals capable of being stored, transferred, combined, compared, and otherwise manipulated.

Useful devices for performing the software implemented operations and functions of the present invention include, but are not limited to, general or specific purpose digital processing and/or computing devices (e.g., a portable computer) structured and configured to perform the specific functions and features, which devices may be standalone devices or part of a larger system. These devices may be selectively activated or reconfigured by a program, routine and/or a sequence of instructions and/or logic stored in the devices, to perform the functions and features disclosed herein. In short, use of the methods described and suggested herein is not limited to a particular processing configuration.

For purposes of illustrating the principles of the present invention and not by limitation, the present invention is described herein below by reference to the exemplary NeuroTherapeutic Wellness System (NTWS) developed by Tinnitus Otosound Products, Inc. However, it is understood that the present invention is equally applicable to systems of other configurations embodying the invention, without departing from the scope and spirit of the present invention.

Overview

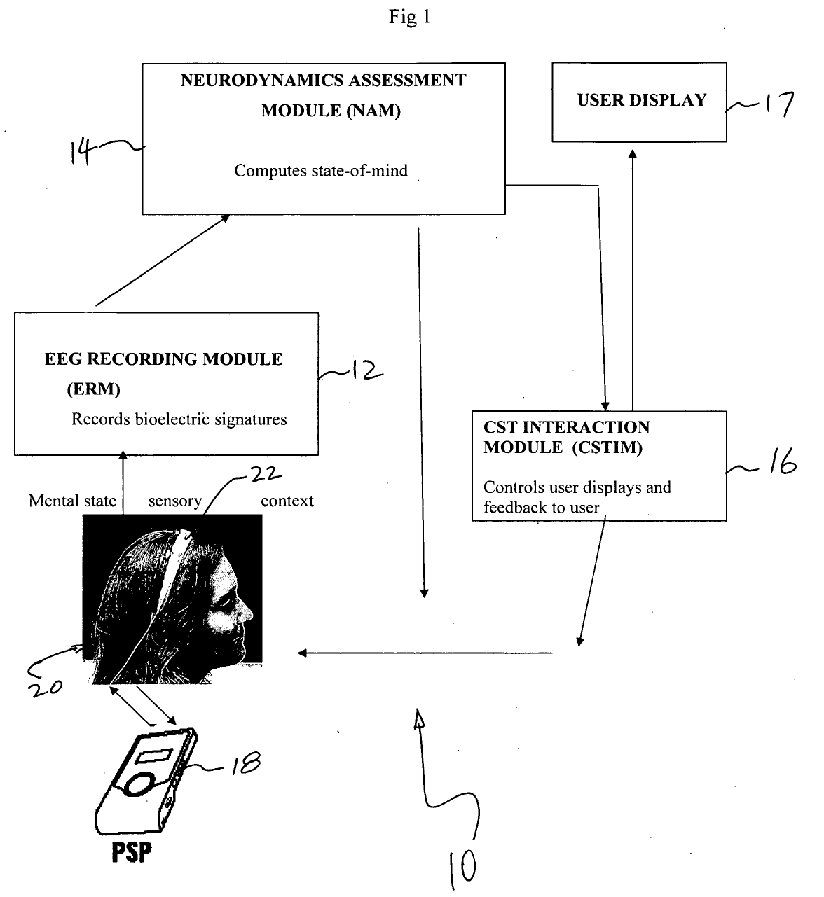

The present invention comprises a means and method for inducing a temporary physiological state-of-mind (e.g., increased relaxation) to effect persistent changes to the cognitive-emotive profile of the individual (e.g., reduce internal tinnitus). Capable of rapidly recognizing the functional significance of the mental and brain function, the invention represents a unique approach to neurofeedback and “mental-state” therapy. The invention makes possible sensitive management of types and levels of therapeutic and non-therapeutic interventions. The sense- and mental state-awareness responses, integrated into a two-way (i.e.,. bi-directional) feedback system using a dynamic interface with intelligently controlled thresholds, comprise a novel adaptive NeuroTherapeutic Wellness System™ (NTWS). (See FIG. 1.) The invention takes into account details of multi-variate and nonlinear dynamics and database templates to more accurately compute the user's “state-of-mind.” It then utilizes this “state-of-mind” to drive therapeutic and non-therapeutic stimulus intervention. By way of a “combinatorial stimulation sequence” approach that uses customized sounds, the present invention creates a fine-tuned and well-controlled process. The significance of this interactivity is a prolonged change in the individual's cognitive-emotive profile.

One simple embodiment of the adaptive therapeutic intervention described by this invention is facilitation of the effectiveness of customized sound therapy for its application in tinnitus relief. It involves coupling tinnitus therapy with real-time relaxation changes in the behavioral profile of a user. The present invention provides a user interface device that is held by a user, and can be clipped to a belt or worn around the neck on a chain. The disclosed device, which resembles an ipod or MP3 music player, is designed to playback customized sounds previously recorded on the player, as well as to capture EEG data regarding a user's levels of relaxation. As used herein, customized sounds refers to the characterization of the internal tinnitus experience of the user. These sounds are played back via earphones to both ears of the user. EEG data refers to the electrical brain activity or changes in voltage recorded by dry, non-contact electrode sensors placed on the scalp of the user. Wireless EEG sensors are attached to the same earpiece that hooks on to the ear and provides the acoustic sound to the user. Thus, the interface device is used to provide both habituation therapy for tinnitus, whereby the playing of the customized sounds leads to a decrease in the tinnitus experience, and relaxation therapy whereby the user learns to relax through a closed-loop visual feedback of his state of relaxation. The captured electrophysiological data from the scalp of the user is in the form of analog data, which allows the input device to be used as an interactive device with a computer program stored in the player. The computer program converts the analog to digital data, extracts features of the EEG and converts those features into a visual display on the player. Neurofeedback devices such as this measure changes in the EEG and display these effects for the user/patient to see. The patient then attempts to exert volitional control over these measured parameters. The tinnitus sound therapy and relaxation therapy can be engaged independently. However, this invention makes it possible to couple the two in such as way as to drive or control the customized sound therapy by the level of relaxation being experienced by the user. For example, habituation sounds would not play if the user's state is one of high anxiety but only below a certain threshold level of relaxation.

Further embodiments of this idea involve a more complex analysis of the state of mind of the user (rather than just the level of relaxation) as that which drives the sound therapy. Bioelectric signals recorded at the scalp provide a window into the complex dynamics of brain activity related to sensation, motor, and cognitive behavior. The use of brain signals to assess sensorimotor (“sense awareness”) and psychological (“mental state awareness”) dimensions would provide the front end to such an adaptive therapeutic system. Furthermore, this invention makes it possible to assess the bodily environment (e.g., HR, GSR, and EOG) in which a behavior occurs (“context awareness”). Hence, the multi-dimensional, non-linear combination of sensation, mental state-, and context-awareness information provides a very realistic bioelectric snapshot of an individual's “state-of-mind.”

NeuroTherapeutic Wellness System™ (NTWS)

Therapeutic intervention that dynamically and in real time adapts to a person's response to the therapy provides a level of care that is more effective than traditional methods. The use of brain bioelectric signals to index the level of physical or psychological stress and anxiety as well as well-being (“wellness indices”) provides the front end to such an adaptive system. Furthermore, the actuation of electronic and/or mechanical therapeutic devices in real time by the ongoing bioelectric “wellness indices” provides sensitive management for the levels of sound based stimulation therapy that may be required at different time points during such an intervention. These types of therapy-user-response-neurofeedback interactions provide for an adaptive NeuroTherapeutic Wellness System™ (NTWS) that increases the degrees of freedom that a patient has over therapeutic intervention and gives an individual control over the level of stimulation therapy required to enhance wellness.

A substantial number of studies have shown that quantitative EEG (QEEG) can be an effective way to determine unique patterns of electrical activity in a variety of common medical and mental disorders, from fibromyalgia to learning disabilities and attentional deficit hyperactivity problems. These phenotypic characteristics are present when symptoms are present and disappear when effective treatment is applied and symptoms are abated. In one embodiment, the NTWS includes apparatus, devices, components, processes and methods for providing therapy for disorders such as tinnitus (one or more of these may be part of the tinnitus therapy applied to the patient). The NTWS is used by a person (e.g., a qualified healthcare professional, such as an otolaryngologist, an audiologist, or other qualified professional, or individual patients themselves with sufficient training) to provide therapy to a patient.

As shown in FIG. 1, the NTWS 10 in accordance with one embodiment of the present invention comprises three primary modules: an EEG Recording Module (ERM) 12, a Neurodynamics Assessment Module (“NAM”) 14 and a CST Interaction Module (“CSTIM”) 16. The features and functionalities of these modules are implemented by software routines and processes. In one embodiment, these modules may be implemented into a Portable Sound Player (PSP). In another embodiment, the PSP 18 may be part of or operatively interact with the NTWS 10, or the modules may be implemented in two or more devices operatively and functionally coupled wirelessly (e.g., Wifi, Bluetooth, IR, etc.) or by wire (e.g., USB, Ethernet, etc.). A display 17 (e.g., on the PSP 18) provides display of EEG data and other information to the user. The modules may be distributed over a LAN or WAN (e.g., the Internet), to allow user access to module(s) or components thereof for carrying out the functions, features and processes in accordance with the invention described herein. For example, the NAM 14 may be accessible via the Internet from an Application Service Provider (ASP).

The ERM 12 provides a means for recording the bioelectric signatures of an individual. The ERM 12 is incorporated as part of an integrated headset. The ERM 12 consists of a high precision, low interference cap containing EEG sensors and data-acquisition circuitry with high-bandwidth communications supporting free motion and continuous use, during self-controlled and guided-mode interventions and monitoring when the individual is alone. The ERM 12 device utilizes special dry electrodes, optimized to record the maximal signal with the fewest number of recording sites and which is easy to put on and operate by anyone. It also includes circuitry built into the headset that ensures excellent signal-to-noise and relatively noise- and artifact-free EEG signals.

EEG is detected and digitized by an analog to digital board at a sampling rate that varies with functionality. The bioelectric signal, and/or derived signatures, can be transmitted to a remote receiver that is connected to a portable microprocessor. Communication between components of the system and other external modules is bi-directional and options for its implementations make it network- and internet-ready.

The NAM 14 module assesses the multi-dimensional, non-linear combination of sense- and mental state-awareness information from the central and peripheral bioelectric signals and provides a real-time snapshot of the individual's state-of-mind. This module is integrated into an assessment regime that involves simultaneous measurements of multiple components of the brain signals to track stimulus depth, effectiveness and real-time cognitive, emotional, and behavioral responses relevant to the assessment regime. The NAM 14 incorporates a portable data capture and analysis system with real-time monitoring capabilities supported by a suite of post-processing software modules for neurological, psychophysical, and psychological assessments. The module acquires multiple brain signals from individuals using real-time analog-to-digital conversion and analysis of signals via the ERM 12 headset and incorporates the use of a dedicated microprocessor-based scientific software, which resides in the microprocessor computer for computerized analysis. The signals are converted into a digital stream and supported by the microprocessor-based software and database processing capabilities, the NAM 14 compares the current physiological state (“state-of-mind”) to a set of templates stored in normative databases and extracts a temporary, multi-dimensional “cognitive-emotive profile” that reflects a more accurate state of mind of the user. This profile contains the individual, integrated electrophysiological indices and their associated boundary conditions and may be updated as necessary to be customized for individual users.

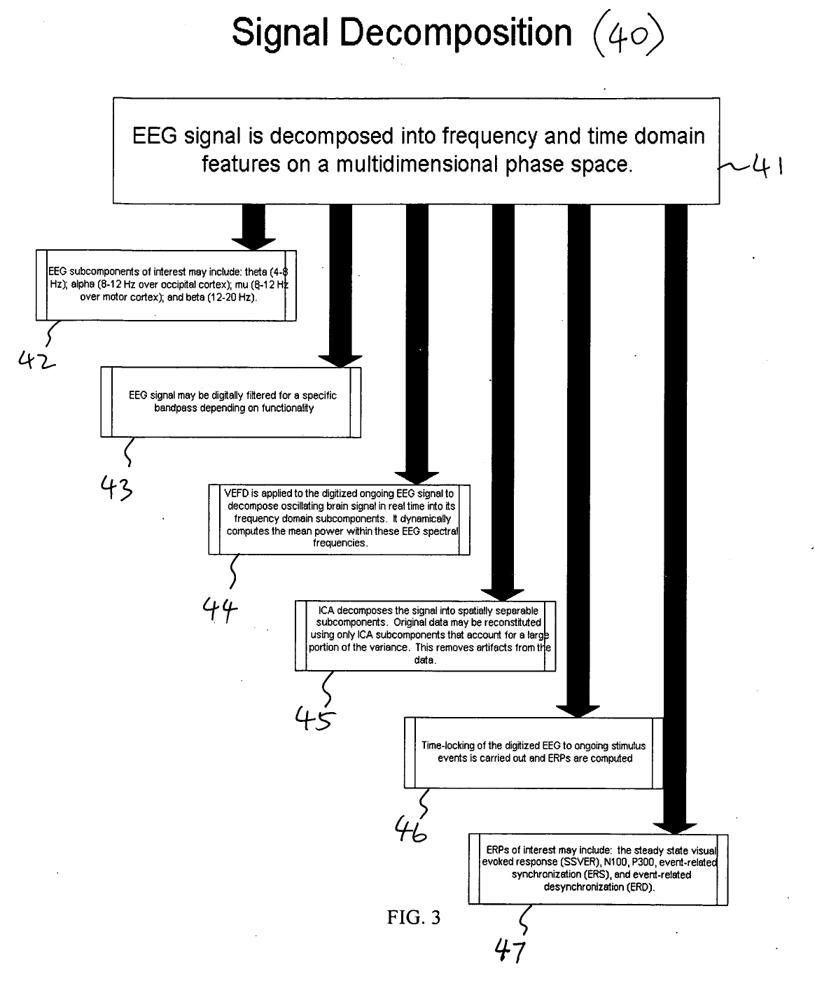

The NAM 14 also includes digital filtering, signal averaging, real-time power spectrum analysis, and calculation of the ongoing power in different frequency bands. It provides data collection, real time analysis, and delivering of output based on the result of the analysis. As depicted in FIG. 3, the digitized EEG signal is decomposed into frequency and time domain features on a multidimensional phase space. Frequency and time domain subcomponents are analyzed using a variety of techniques including Variable Epoch Frequency Decomposition (VEFD), Fast Fourier Transform, Event-Related Potentials (ERPs), Independent Component Analysis (ICA), Time-Frequency Expansion, and/or Feature Coherence Analysis. The EEG subcomponents of interest include EEG rhythms, such as mu (7-13 Hz over sensorimotor cortex), theta (4-8 Hz); alpha (8-12 Hz over occipital cortex); and beta (12-20 Hz). They can also include time-locked responses to external events, or event-related potentials, such as the traditional N1, P3, or the steady state visual evoked response (SSVER).

The brain signal is digitally filtered for a specific bandpass depending on which of these signals is being used. In most applications, ICA decomposes the signal into spatially separable subcomponents in order to maximize the signal-to-noise response and allow for multiple control signals. That is, original data may be reconstituted using only ICA subcomponents that account for a large portion of the variance in the signal. This removes blinks and eye movement artifacts from the data. Using ICA to “clean” the data in real time increases signal-to-noise ratio, making the relevant signal easier and faster to detect by a pattern recognition system. Identification of multiple independent control signals in the EEG makes capturing a more realistic state of mind possible. Decomposed EEG data are subjected to a state discriminant analysis to identify “feature” clusters that are most reliably different between different conditions. Feature clusters represent patterns of electrical activity that occur across the scalp and that are linked to specific thought patterns. They may be analyzed using waveform analysis, distribution function analysis, Fuzzy logic, discriminant optimization, and/or other approaches. The outcome of this analysis is the creation of a BCI Feature Map (BFM), which is represented as a set of parameters, components, functions, and criteria. BFMs are constituted as input into a pattern recognition system, which may be expressed in the form of a neural network, genetic algorithm, Kohonen network, Fuzzy neural net, or Markov model. The output of the pattern recognition system is a set of activations or BCI Neural Activations (BNAs). BNAs are derived from adaptive combinations of discriminant brainwave features in space, time, frequency, and phase that come together to maximize the contrast between conditions.

The NTWS 10 includes a control module, in the form of the CSTIM 16 in accordance with one embodiment of the present invention. The CSTIM 16 can engage with the customized sound algorithms to make CST state-dependent. That is, patients will receive CST as a function of their state-of-mind. For example, below a specified level of anxiety or stress, CST would be provided but not if the level of anxiety/stress exceeds a specified threshold. The CSTIM 16 also receives the feedback from the Neurodynamics Assessment module to allow adjustment of the combination of sounds that would be presented. The CSTIM 16 is software implemented, and may be integrated into the PSP 18 with the ERM 12 hardware, which can be worn by an individual. The CSTIM 16 can be engaged to allow CST to be state-dependent or disengaged if CST is to be used regardless of the state of the patient.

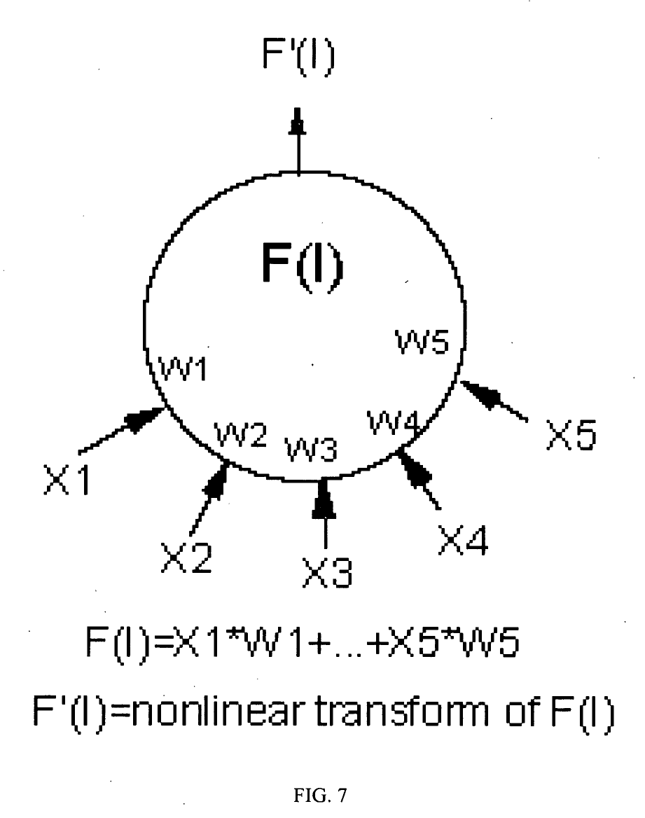

The software implemented routines and processes supporting the various components and modules include a library of data analysis routines, from which bioelectric indices are obtained from the analysis of spontaneous, event-related, and steady state brain responses as well as other naturally occurring bioelectric activity. As depicted in FIG. 7, the indices are mapped to effect control of the CSTIM 16 device, such as to adjust the combination and levels of stress. Thus, the CST software function becomes “sense-aware” and “mental-state-aware.”

The “sense-awareness” and “mental state-awareness” are integrated into a part of the boundary conditions of the resulting cognitive-emotive profile. The present invention provides for software implemented functions that allows for flexible mapping of this information. The sense- and mental state-awareness capabilities are activated when the CSTIM 16 is enabled.

The system software implemented tool provides a mapping capability with the ability to weigh variables and to apply them in appropriate calculations and to capture them in computer files for post-processing. A flexible embedded scripting language in the tool, and user memory in the main application, enables simple, limited conversions of data formats and conditional statement control that can run in real-time for appropriate system interfacing. The computational output is also used to provide visual feedback information to the subject and to adapt the data analysis/extraction process implemented by algorithm to best match the incoming data (adaptive data extraction). Once a pattern of brain activity is identified, the BNAs are dynamically mapped onto a set of microprocessor-based system commands that reset the combination and levels of stimulation.

In addition, a biofeedback signal is provided to the user. The dynamic mapping also allows advantages in several “open-loop” situations where the user does not necessarily need to detect and employ feedback to utilize CST. The system can be used in self-controlled mode, but also in guided-mode with cooperative and uncooperative individuals, such as in medical settings. In the self-controlled mode, the system increases the degrees of freedom that a person has over medical or non-medical treatments and gives an individual control over the level of stimulation required to change a “cognitive-emotive profile.”

From the above, it is apparent that the present invention represents a unique, approach to neurofeedback and “mental-state” therapy. It takes into account details of multi-variate, nonlinear dynamics that more accurately reflect the user's “state-of-mind” and utilizes it to drive the therapeutic and non-therapeutic stimulus intervention. Thus, the present invention effectively integrates a “combinatorial sound-based stimulation sequence” with a “combinatorial EEG recording sequence” to create a fine-tuned and well-controlled process. The significance of this interactivity is a prolonged change in the individual's cognitive-emotive profile.

This invention may be deployable over diverse areas of human activity, including enhancing work performance, such as operator speed and accuracy, alternative learning techniques, military applications such as debriefings and interrogations, and rehabilitation for violent behavior and addictions of various types. Specific applications include, but are not limited to, monitoring brain disorders, ameliorating specific disorders (such as sleep disorders, mood disorders, OCD, attention-deficit and other attentional deficits), monitoring and inducing alertness and cognitive readiness in individuals to ensure they perform their jobs safely and adequately, having the ability to acquire information and to evaluate the validity, truth or falsity of such information, and aid in relaxation, motivation, or induction of other specific cognitive-emotive states desired by the user.

In one embodiment, the present invention provides an intelligent control interface, and a method that utilizes bioelectric signals to control sound-based therapy for central auditory processing disorders, such as tinnitus. The interface comprises means for acquiring the bioelectric signals of a user, which are converted into a digital stream and processed to define a cognitive-emotive profile or “state of mind” of the user. Incorporating microprocessor-based software and database capabilities, the interface then dynamically maps the cognitive-emotive profile onto customized sound therapy processes implemented algorithms that create a specified sound for each patient. With reference to the figures, the invented system is now described in detail.

There are several major functional components of the system of the present invention: 1) acquisition of bioelectric activity; 2) real-time analog-to-digital conversion; 3) preprocessing and data analysis; 4) learning algorithms; 5) pattern recognition; 6) signal mapping of microprocessor commands; and 7) closed-loop feedback.

Bioelectric Activity Data Acquisition

The intelligent control interface of the present invention incorporates sensors which are adapted for the detection of bioelectric signals. These sensors are commercially available and they require a minimum of preparation and use (e.g., dry, non-contact electrodes). These sensors are proximally placed on the scalp and on the body of the user to record the signals without user preparation of the scalp. The sensors can be incorporated as part of a high precision, low interference headset, with disposable gel-filled inserts or saline-based electrodes, with built-in amplifiers. This ensures excellent signal-to-noise and relatively noise- and artifact-free signals. Analog-to digital (A/D) conversion is performed by dedicated converters, which are also built into the headset. The signals can be transmitted by wireless means to a remote receiver that is connected to a portable microprocessor. A built-in driver interface permits the data acquisition system to communicate with the remote receiver. These features allow an individual to be untethered to the computer or electronic device running the virtual environment, that is they can walk around freely. The level of noise that typically interferes with such natural body movements are significantly reduced.

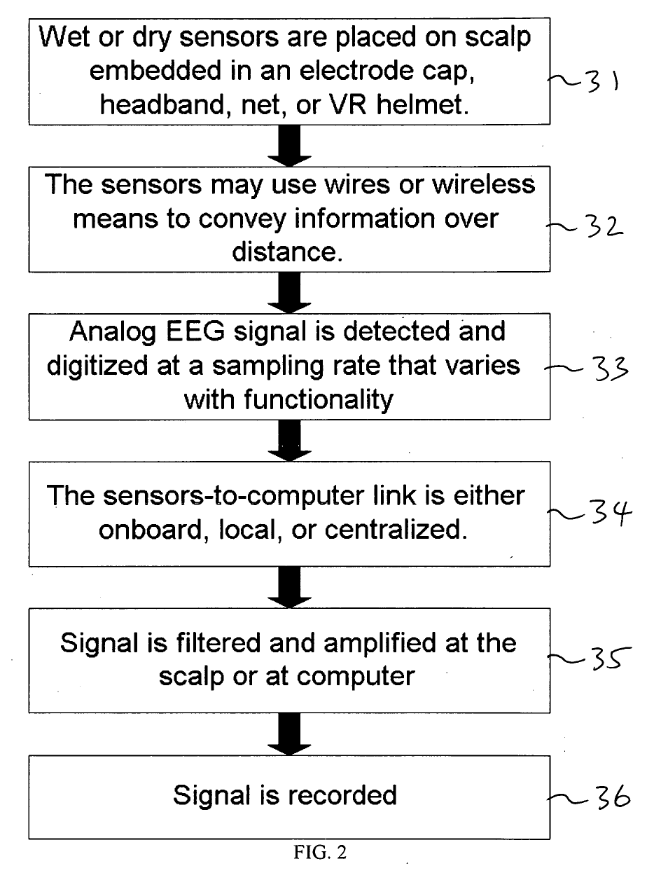

FIG. 2 shows the diagrammatic sequence of steps involved in the recording stage 30 by the ERM 12. Referring also to FIG. 1, a user 20 carries a sensor device 22 on the scalp (at 31). The sensors 22 may be embedded in commercially available conventional electrode caps, headbands, nets, virtual reality (VR) helmets, or other means (at 32). The sensors 22 use wireless means, either radio frequency (RF) or infrared data association (IrDA) means using Object Exchange Protocol (IrOBEX) to convey information to a recording microprocessor (e.g., in the PSP 18). The sensors-to-microprocessor link can be onboard (i.e., both sensors and microprocessor are on the body), local (the sensors and microprocessor within a defined distance of each other); or centralized (the sensors and microprocessor at a very large distance from each other) (at 34). Bioelectric signals are detected and digitized by an analog to digital board at a sampling rate that varies with functionality (at 33). As an example, the use of spontaneous EEG rhythms requires fast sampling rates, while the use of event-related potentials requires slower sampling rates. Analog signals are filtered (bandpassed) and amplified (either at the scalp or remotely at the recording microprocessor), and digitized (at 35). The digital signal is recorded, e.g., in the PSP 18 (at 36).

For purposes of illustration, two types of brain signals are recorded and analyzed for computing a “sense awareness” index and a “mental state awareness” index These include spontaneous EEG rhythms, time-locked responses to external events and steady state visual evoked responses Additionally, peripheral signals from the heart, galvanic skin response (GSR), and electrooculogram (EOG) are also recorded and analyzed to compute a “context awareness” index. “Context-awareness” is activated when a person is engaged in performing an instrumented and well-characterized procedure from which task-relevant parameters can be captured during performance.

Real-Time Analog-to-Digital Conversion

The ERM 12 of the NTWS 10 provides for the real-time analog-to-digital conversion and analysis of bioelectric signals. It incorporates a dedicated microprocessor-based scientific software system, which resides in a microprocessor computer. The software system includes a library of data analysis routines for processing spontaneous, event-related, and steady state brain responses and peripheral autonomic signals, including digital filtering, signal averaging, real-time power spectrum analysis, and calculation of the ongoing power in different frequency bands. It provides data collection, real time analysis, and delivering of computational output of the analysis. The output is provided to the user or be applied to adapt the data analysis/extraction algorithm to best match the incoming data (adaptive data extraction).

The present invention generates two types of outputs for resource control-digital (e.g. on/off firing of a weapon, turning devices on/off, or sending digital information to a networked associate), and analog (graded), via digital-to-analog (D/A) converters that are built into the headset. The system analyzes incoming data in real time, as it is acquired, and then triggers a command based on the result of the analysis. This system uses a simple uniform structure for data representation with the same data format for both input and output data (the raw incoming data and the results of an analysis) to ensure, among other things, that the output of one computation can be used as an input for another. Data already collected and pre-processed can be reused and analyzed in a different way. The system also supports the export of data in a format that can be used by other microprocessor programs to perform independent component analysis or neural net analysis.

Preprocessing and Analysis of the Data (Signal Decomposition)

In the signal decomposition process 40 (e.g., by the ERM 12 or NAM 14 or both) as depicted in FIG. 40, the digitized bioelectric signals are decomposed into frequency and time domain features on a multidimensional phase space (at 41). Frequency and time domain subcomponents are analyzed using a variety of techniques including Variable Epoch Frequency Decomposition (VEFD), Fast Fourier Transform, Event-Related Potentials (ERPs), Independent Component Analysis (ICA), Time-Frequency Expansion, and/or Feature Coherence Analysis. The EEG subcomponents include EEG rhythms, such as mu (7-13 Hz over sensorimotor cortex), theta (4-8 Hz); alpha (8-12 Hz over occipital cortex); and beta (12-20 Hz) (at 42). They also include time-locked responses to external events (at 46), or event-related potentials, such as the traditional N1, P3, or the steady state visual evoked response (SSVER) and peripheral autonomic signals (e.g., HR, GSR, and EOG) (at 47). The signals are digitally filtered for a specific bandpass depending on which of these signals is being used (at 43).

In certain applications, VEFD is applied to the digitized signal in real time to decompose oscillating rhythms into their frequency domain subcomponents (at 44). For example, in order to determine how tired a user is, the system examines the level of alpha and beta activities in the EEG. In the present invention, VEFD provides for a robust method of brain signal detection.

In other applications, ICA decomposes the signal into spatially separable subcomponents in order to maximize the signal-to-noise response and allow for multiple control signals (at 45). Original data may be reconstituted using only ICA subcomponents that account for a large portion of the variance in the signal. This removes blinks and eye movement artifacts from the data. Using ICA to condition the data in real time increases signal-to-noise, making the relevant signal easier and faster to detect by a pattern recognition system. The use of ICA thus provides a solution to the problem of blind source separation. Blind source separation is analogous to a situation posed by recording bioelectric signals at multiple sites where the signal at any recording site (be it a satellite, microphone, or electrode) is assumed to consist of a combination of numerous overlapping sources. The locations of these sources are unknown, and the objective is to isolate the contribution of each of these independent sources based on the observed data at each site. Identification of multiple independent control signals in the input makes simultaneous control of multiple functions feasible in the present invention. For example, in a virtual game environment it allows an avatar to jump, fire, and signal to others all at the same time.

Learning Algorithms

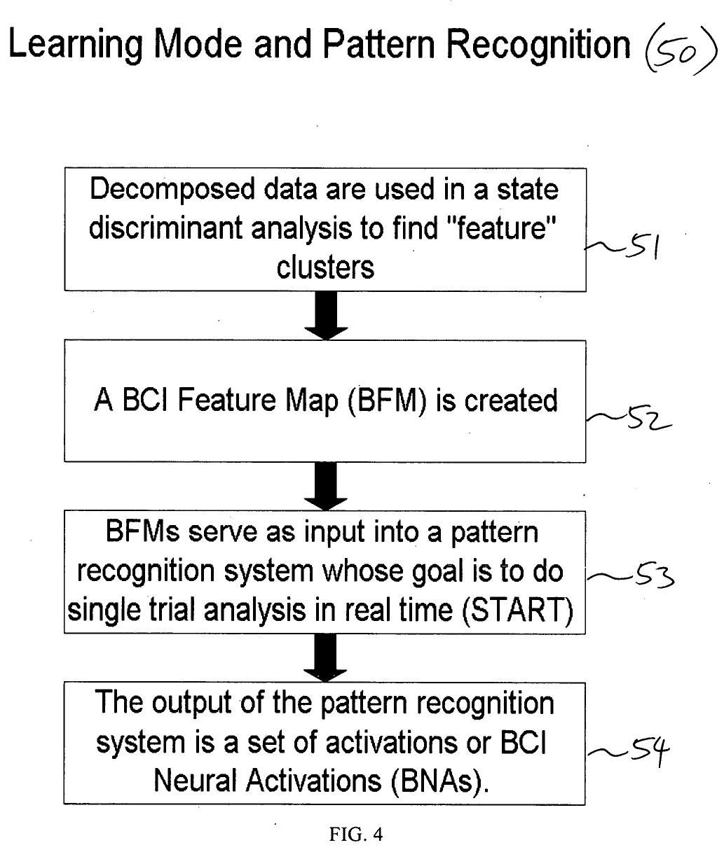

Learning and pattern recognition processes 50 are undertaken by the NAM 14 for example, are schematically depicted in FIG. 4. The NAM 14 of the NTWS 10 provides a means. whereby decomposed EEG data are resolved by way of a state discriminant analysis to identify “feature” clusters that are most reliably different between different conditions (at 51). Feature clusters represent patterns of electrical activities that occur across the scalp, which are linked to specific motor or non-motor thought patterns. For example, when a user sees a novel image on the screen, a large positive-going voltage can be detected over the middle of the scalp approximately 300 milliseconds after the onset of the novel image. This would be a feature cluster identifiable in the discriminant analysis. This may be accomplished using: waveform analysis, distribution function analysis, Fuzzy logic, and/or discriminant optimization. The outcome of this analysis is used to define a BCI Feature Map (BFM) (at 52), which is represented as a set of parameters, components, functions, and criteria.

Pattern Recognition

A plurality of the BFMs are constituted as input into a pattern recognition system (at 53), which may be expressed in the form of a neural network, genetic algorithm, Kohonen network, Fuzzy neural net, or. Markov model. The output of the pattern recognition system is a set of activations or BCI Neural Activations (BNAs) (at 54). BNAs are derived from adaptive combinations of discriminant brainwave features in space, time, frequency, and phase that come together to maximize the contrast between conditions.

The present invention provides classification of patterns of brain activity in real time. Neural networks, or other pattern recognition systems, are used to determine underlying functional relationship between power spectrum fluctuations as they relate to changes in thought patterns. Employing a neural network classifier as a structure with modifiable parameters is of benefit for the following reasons: (a) underlying relationships which are assumed to exist, are not known, and are to be found; (b) by supplying the neural network with training sets obtained from recordings on single subjects, the network “learns” individual patterns; and (c) the method can be adjusted to correspond to the results obtained by visual inspection of different experts. This approach has proven very effective in recognizing complex patterns such as the ones produced by sensor arrays in actual environmental conditions. They have proven to be useful in numerous tasks involving categorization of bioelectric patterns. In the present invention, these properties allow for the rapid and reliable recognition and learning of brain patterns that are consistently mapped to functions in an environment associated with a device. Thus such a device can be customized to the user with rapid recognition of the user's meaningful brain patterns.

Mapping Signals to Microprocessor Based System Commands and Closed-Loop Feedback

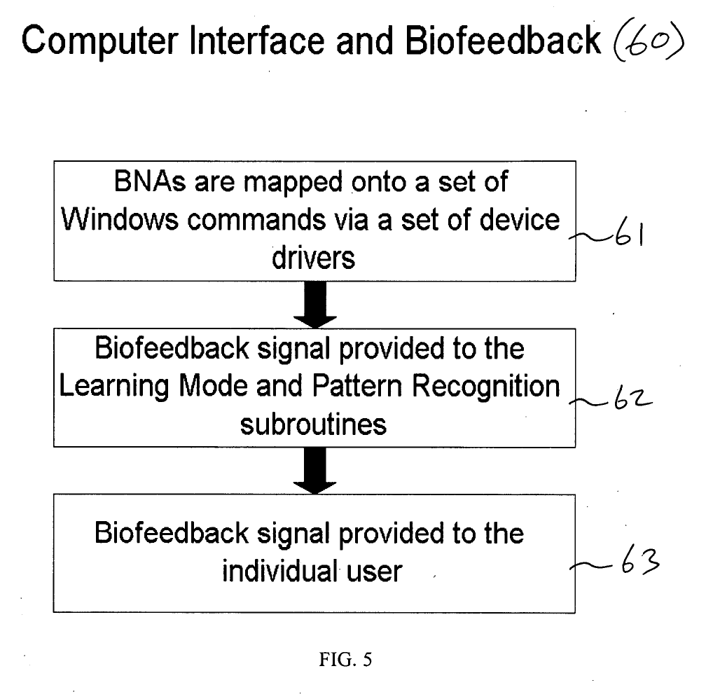

According to the process 60 depicted in FIG. 5, once a pattern of brain activities is identified, the BNAs can be dynamically mapped (e.g., by the NAM 14) onto a set of microprocessor-based system commands (at 61). By way of examples, the commands may include Windows commands for keyboard command, cursor movement control, file operation, and protocol control. Biofeedback signals are provided to the Learning Mode and Pattern Recognition subroutines (at 62). In addition, a biofeedback signal can be provided to the user (at 63), e.g., with the NAM 14 interacting with the CSTIM 16. The dynamic mapping has advantages in several “open-loop” situations where the user does not necessarily need to detect and employ feedback to achieve robust assertion of a desired control, such as to reduce stress. An open-loop feedback system, such as this, provides enhanced freedom for the individual to exert control and can increase the scope of activities to be used.

The NTWS 10 may also employ Neural Networks (NNs) (e.g., by the NAM 14), which provide further supports in pattern recognition and robust classifiers, with the ability to generalize in making decisions about imprecise input data. In addition, the NNs may also be applied to control problems, such as in the present invention, where the input variables are measurements used to drive an output actuator, and the network learns the control function.

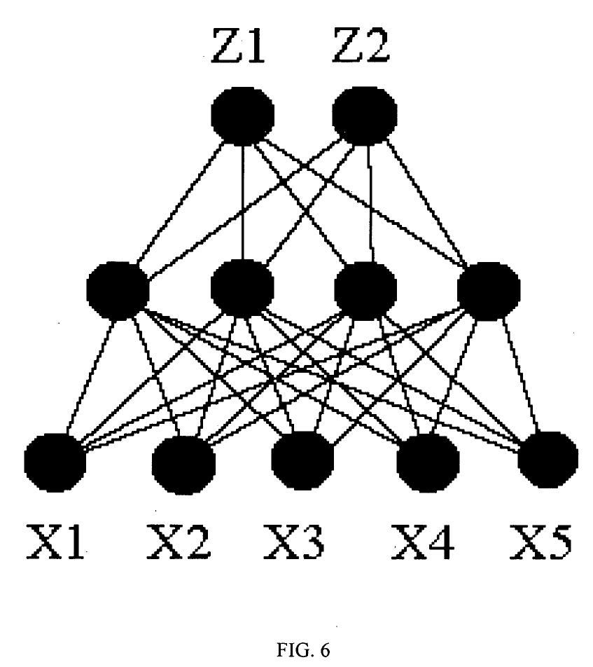

As an example, the structure of a neural network is represented in FIG. 6 where the bottom layer represents the input layer, in this case with 5 inputs labeled X1 through X5. These inputs comprise varying levels of three categories of brain signals recorded, which can be extracted from different recording sites. In the middle of the network is the hidden layer, with a variable number of nodes. It is the hidden layer that performs much of the work of the network. Each node in the hidden layer is fully connected to the inputs. The hidden layer is where the network learns interdependencies in the model. FIG. 7 illustrates predefined relationship for the manipulation and translation of the brain signal into the output functions associated with the output layer, which comprises at least one node. By way of an example, the player in a video game may be trying to predict where an enemy soldier will appear next (output) based on past appearances, the attentional gaze of the user, and the warning signal from a friendly soldier (input). The computation to determine these interdependencies involves a two-layer feed-forward neural network. It consists of two layers of weights (the neural network name for the model parameters) and two (or three) layers of “neurons” (or units). The first layer of neurons is not usually counted as a layer: It is the input to the neural network. The second layer is the hidden layer. The neurons in this layer have an activation function, and it is necessary for the non-linearity of neural network that this activation function is non-linear. The final layer is the output layer. These will also have an activation function. This might be linear or non-linear. With “x” as the input, “y” as the output, with “v” as the first layer of weights (the input-to-hidden weights) and “w” as the second (the hidden-to-output weights) and with i, h, o, and p as the indices for the input, hidden and output neurons, and the examples, respectively, we get the following neural network function:

$${y}_{o}^{p} = {g}^{o} ( {\sum }_{h}^{{n}_{h}} {w}_{ho} {g}^{h} ( {\sum }_{i}^{{n}_{i}} {v}_{ih} {x}_{i}^{p} + {v}_{h 0} ) + {w}_{o 0} )$$

Here, g^{o} and g^{h} are the activation functions and vₕ₀ and wₒ₀ are the biases.

The activation function is usually of the signoidal type and we use the hyperbolic tangent. In connection with classification the output activation function is this hyperbolic tangent to get a restricted output that can be interpreted as a probability. In connection with regression the output activation function is linear.

During training, the network is repeatedly shown observations from available data related to the problem to be solved, including both inputs (the X1 through X5 in FIG. 6) and the desired outputs (Z1 and Z2 in the diagram). The network tries to predict the correct output for each set of inputs by gradually reducing the error (backpropagation of error algorithm). There are other algorithms for accomplishing this (learning vector quantization, radial basis function, Hopfield, and Kohonen), but they all involve an iterative search for the proper set of weights (the W1-W5) that will do the best job of accurately predicting the outputs.

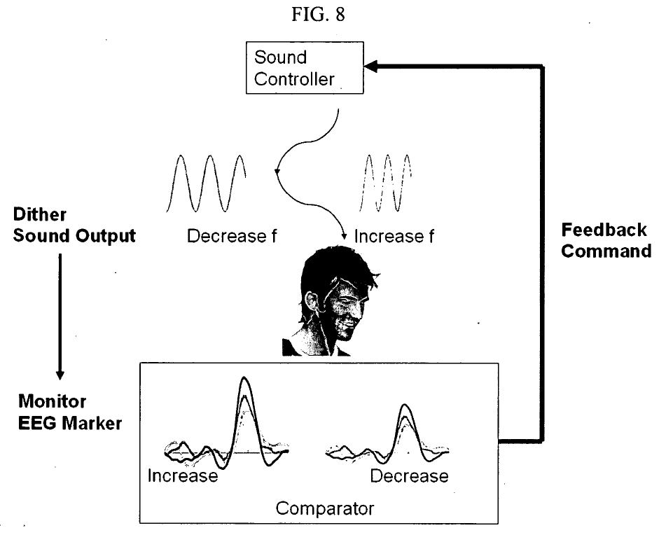

Referring to the schematic diagram in FIG. 8, the controlling system for driving neural activity changes. The example shown is for tinnitus, however, using a suitable neural marker, many nervous system anomalies could be addressed by this system. The core is a subject listening to sounds who is simultaneously having EEG recorded. In the “comparator”, a neural signature is monitored. In the tinnitus example, the signal response of the auditory cortex to a sound matching the tinnitus stimulus is the “EEG Marker”. If the comparator detects that the EEG marker is increasing, i.e. that the brain's response is getting worse, then the feedback signal to the “sound controller” is to “Dither” (i.e. vary) the sound the subject is listening to. Dithering in this circumstance could be a variation of the frequency of the sound stimulus. If the comparator detects that the neural marker is changing in the correct direction (decreasing in this example), then no changes are signaled to the sound controller by the feedback pathway.

Thus, from the above, it is apparent that the present invention represents a unique, novel, more natural, intuitive, and hands-free means of communicating with and controlling resources in a virtual reality world, which takes into account details of nonlinear neurodynamics and reflects the user's state of the mind.

Portable Sound Player (PSP)

The invention is operational with numerous general purpose or special purpose computing devices, system environments or configurations. Examples of well known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, embedded systems, distributed computing environments that include any of the above systems or devices, and the like.

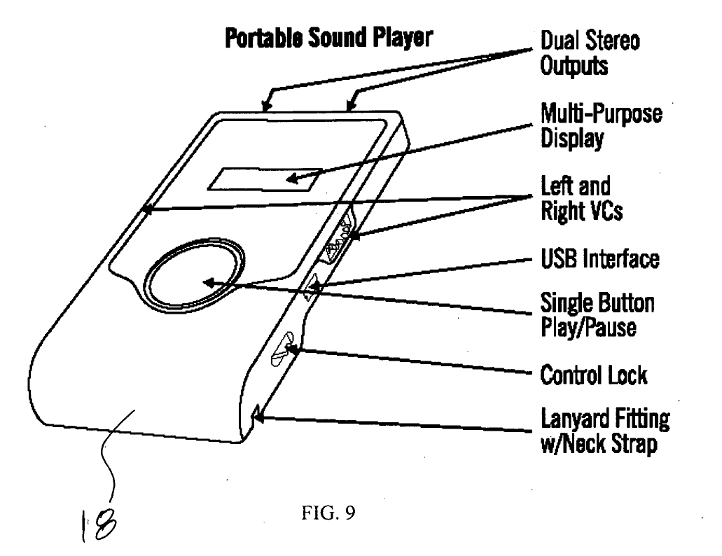

Referring to FIG. 1, ISP 18 may include the NTWS 10. An embodiment of a PSP is disclosed in earlier filed Provisional Patent Application No. 61/001,209 and U.S. patent application Ser. No. 12/215,385, which had been assigned to the assignee of the present invention, and which had been fully incorporated by reference herein. Such PSP can be adapted with the necessary hardware, software, firmware implementations to perform the functions, features and operations of the present invention disclosed herein (e.g., the NTWS). For example, the PSP 18 shown in FIG. 9 may include a processing unit (e.g., a CPU, microprocessor, ASIC), volatile and nonvolatile memory (e.g., RAM, ROM, EPROM), system BIOS, removable and non-removable data media for storage of information such as computer readable instructions, data structures, program modules or other data (e.g., Secure Digital (SD) card, mass storage (e.g., hard drive)), communication/data ports/interfaces (e.g., USB, Ethernet, IR, display, audio), user I/O (e.g., LCD display, function keys, audio output), and a system bus that couples the various system components. In a networked environment, program modules or portions thereof, may be stored in the remote memory storage device.

As noted in the earlier filed applications, the PSP has several requirements (such as volume limitation, playback session logging) that are not found in a typical audio player. Unlike a general audio player, the PSP does not need to produce sound at high volume levels, such as those suitable for general music listening. Further details of the PSP are disclosed in the earlier filed applications. For adaptation of the PSP for the present invention, it includes the additional functionality of the NTWS disclosed herein.

In one embodiment, the PSP 18 includes the following structures, features and functions:
A convenient on-off control to preserve battery life.
A multi-purpose display.
A long battery life of at least 8 hours of playback between charges.
A convenient play-pause button.
A control hold (lock-out) button to prevent accidental change of settings.
Continual, repeated playback of a single recorded sound of a preset duration (e.g., at least 5 minutes duration).
Sterophonic playback to allow for differential playback in each ear.
A balance control to allow the relative loudness in each ear to be adjusted.
A convenient volume control and display giving a numerical readout of volume level.
Playback volume limitation limited to a certain volume level.
An internal date and time of day clock to allow internal logging of playback times and volumes.
Internal monitoring software implementations to log playback dates, times, volumes, etc., and the functions and operations of one or more components of the NTWS 10 if incorporated.
USB or other convenient interface to the SMS allowing exchange of sound and logging data, which should include playback times and volumes, and other patient data, such as ID and arbitrary text notes, such as sound specifications.
Transducers for one or both ears, preferably connected wirelessly to the PSP.
Provision for connection to multiple transducers in order to allow monitoring by SMS operator during sound customization or at other times.
Audio requirements: the sound playback should be at high quality, e.g., at a minimum essentially that of standard red book CD audio, i.e., 16-bit linear PCM stereo sampled at 44,100 samples per second per channel. The analog audio output circuitry needs to be of high quality, with noise and distortion characteristics on the order of those of high quality digital music player, such as MP3 players (e.g., the Apple iPod player) or better. The analog audio output needs to be able to drive at least two sets of transducers with independent volume settings (e.g., left and right VCs shown in FIG. 9), allowing simultaneous sound monitoring by both the patient and the CST operator.
Memory requirements: assuming the sound is recorded as standard 16-bit linear PCM stereo audio (1.411 Mbs), the audio storage requirements are on the order of 64 MB. Additional storage for software and data logging may double or quadruple this. Firmware memory requirements are hardware-dependent, and preferably updatable to allow for future improvements.
A lanyard fitting with neck strap, to provide convenience to facilitate patient “wearing” the PSP device with ease, with less interference to the patient's daily routine.
Data input ports for recording EEG bioelectric signatures.

### ALTERNATE EMBODIMENTS

While a PSP providing auditory biosensory feedback stimulus is disclosed herein in accordance with one embodiment of the present invention, a device may be implemented to provide other types of biosensory feedback stimulus, such as visual stimulus (e.g., images), temperature stimulus, motion stimulus (e.g., vibration), offal stimulus (e.g., smell), electric stimulus (e.g., acupuncture), etc.

The process and system of the present invention has been described above in terms of functional modules. It is understood that unless otherwise stated to the contrary herein, one or more functions may be integrated in a single physical device or a software module in a software product, or a function may be implemented in separate physical devices or software modules, without departing from the scope and spirit of the present invention. It will be further appreciated that the line between hardware, firmware and software is not always sharp.

It is appreciated that detailed discussion of the actual implementation of each step that comprises the process is not necessary for an enabling understanding of the invention. The actual implementation is well within the routine skill of a programmer and computer engineer, given the disclosure herein of the system attributes, functionality and inter-relationship of the various software and hardware components in the system. A person skilled in the art, applying ordinary skill can practice the present invention without undue experimentation.

While the invention has been described with respect to the described embodiments in accordance therewith, it will be apparent to those skilled in the art that various modifications and improvements may be made without departing from the scope and spirit of the invention. Accordingly, it is to be understood that the invention is not to be limited by the specific illustrated embodiments, but only by the scope of the appended claims.

1. A system for providing adaptive therapy to a user having a medical disorder, comprising:
a bioelectric sensor module, providing updated neurological information of the user in response to the therapy;
an assessment module, receiving and processing in real time the updated neurological information, to assess and update the behavioral profile of the user;
a therapy module, providing updated biosensory feedback to the user based on the updated behavioral profile of the user from the assessment module, to provide the user therapy for the medical disorder,
wherein the therapy module adapts to the changes in behavioral profile of the user during a therapy session, thereby providing appropriate biosensory feedback in accordance with then current behavioral profile of the user.
